(19) United States
(12) Patent Application Publication
(10) Pub. No.: US 2012/0065508 A1
(43) Pub. Date: Mar. 15, 2012

(54) ULTRASOUND IMAGING SYSTEM AND METHOD FOR DISPLAYING A TARGET IMAGE

(75) Inventors: Olivier Gerard, Horten (NO); Gunnar Hansen, Horten (NO)

(73) Assignee: GENERAL ELECTRIC COMPANY, Schenectady, NY (US)

(21) Appl. No.: 12/878,423

(22) Filed: Sep. 9, 2010

(51) Int. Cl. A61B 8/14

(52) U.S. Cl. 600/443

(57) ABSTRACT

An ultrasound imaging system and method include acquiring ultrasound data of an anatomical structure, generating a live image based on the ultrasound data, and displaying the live image. The system and method include displaying a target image of the anatomical structure. The system and method include comparing the live image to the target image.

### FIELD OF THE INVENTION

This disclosure relates generally to ultrasound imaging and specifically to a system and method for displaying a live image and a target image.

### BACKGROUND OF THE INVENTION

Ultrasound examinations often include the acquisition of ultrasound data according to a specific protocol in order to generate one or more standard views of an organ or anatomical structure. The standard view may include either a single image of the organ or anatomical structure, or the standard view may include multiple images acquired over a period of time and saved as a loop or dynamic image. However, depending on the protocol, it may take considerable skill and time to put the probe in the correct position and orientation to acquire images that are close to the desired standard view. New or non-expert users may experience additional difficulty when trying to acquire images that correspond to one or more standard views. As a result, particularly when the user is a non-expert, it may take a long time to acquire images that correspond to the standard view. Additionally, since the non-expert user may not be able to consistently acquire images of the standard view, results may vary considerably both between patients and during follow-up examinations with the same patient.

Conventional ultrasound systems do not provide a convenient way for a user to determine if the acquisition parameters are correct for a given standard view. Therefore, for at least the reasons described hereinabove, there is a need for an improved method and system for acquiring ultrasound images that correspond with standard views.

### BRIEF DESCRIPTION OF THE INVENTION

The above-mentioned shortcomings, disadvantages and problems are addressed herein which will be understood by reading and understanding the following specification.

In an embodiment, a method of ultrasound imaging includes acquiring ultrasound data of an anatomical structure, generating a live image based on the ultrasound data, and displaying the live image. The method includes displaying a target image of the anatomical structure. The method also includes comparing the live image to the target image.

In another embodiment, a method of ultrasound imaging includes acquiring ultrasound data of an anatomical structure, generating a live image based on the ultrasound data, and displaying the live image. The method includes selectively displaying a target image of the anatomical structure while displaying the live image. The method also includes comparing the live image to the target image in order to validate an acquisition parameter used to acquire the ultrasound data.

In another embodiment, an ultrasound imaging system includes a probe including a plurality of transducer elements, a user interface, a display screen, and a processor. The processor is operably connected to the probe, the user interface, and the display screen. The processor is configured to control the probe to acquire ultrasound data of an anatomical structure. The processor is configured to generate a live image from the ultrasound data. The processor is configured to display the live image on the display screen. The processor is configured to display a target image of the anatomical structure on the display screen in response to an input entered through the user interface.

Various other features, objects, and advantages of the invention will be made apparent to those skilled in the art from the accompanying drawings and detailed description thereof.

### BRIEF DESCRIPTION OF THE DRAWINGS

FIG. 1 is a schematic diagram of an ultrasound imaging system in accordance with an embodiment;

FIG. 2 is a schematic representation of hand-held ultrasound imaging system in accordance with an embodiment;

FIG. 3 is a flow chart illustrating a method in accordance with an embodiment;

FIG. 4 is a schematic representation of a live image and a target image in accordance with an embodiment; and

FIG. 5 is a schematic representation of a target image superimposed on a live image in accordance with an embodiment.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

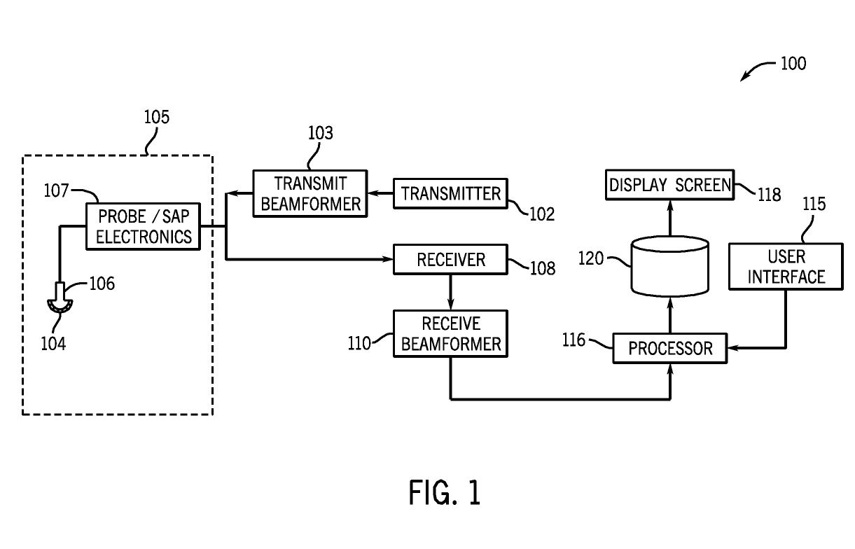

FIG. 1 is a schematic diagram of an ultrasound imaging system 100 in accordance with an embodiment. The ultrasound imaging system 100 includes a transmitter 102 that transmits a signal to a transmit beamformer 103 which in turn drives transducer elements 104 within a transducer array 106 to emit pulsed ultrasonic signals into a structure, such as a patient (not shown). A probe 105 includes the transducer array 106, the transducer elements 104 and probe/SAP electronics 107. The probe/SAP electronics 107 may be used to control the switching of the transducer elements 104. The probe/SAP electronics 107 may also be used to group the elements 104 into one or more sub-apertures. A variety of geometries of transducer arrays may be used. The pulsed ultrasonic signals are back-scattered from structures in the body, like blood cells or muscular tissue, to produce echoes that return to the transducer elements 104. The echoes are converted into electrical signals, or ultrasound data, by the transducer elements 104 and the electrical signals are received by a receiver 108. For purposes of this disclosure, the term ultrasound data may include data that was acquired and/or processed by an ultrasound system. The electrical signals representing the received echoes are passed through a receive beam-former 110 that outputs ultrasound data. A user interface 115 may be used to control operation of the ultrasound imaging system 100, including, to control the input of patient data, to change a scanning or display parameter, and the like.

The ultrasound imaging system 100 also includes a processor 116 to process the ultrasound data and generate frames or images for display on a display screen 118. The processor 116 may be adapted to perform one or more processing operations according to a plurality of selectable ultrasound modalities on the ultrasound data. Other embodiments may use multiple processors to perform various processing tasks. The processor 116 may also be adapted to control the acquisition of ultrasound data with the probe 105. The ultrasound data may be processed in real-time during a scanning session as the echo signals are received. An embodiment may update the displayed ultrasound image at a rate of more than 20 times per second. The images may be displayed as part of a live image. For purposes of this disclosure, the term “live image” is defined to include a dynamic image that updates as additional frames of ultrasound data are acquired. For example, ultrasound data may be acquired even as images are being generated based on previously acquired data and while a live image is being displayed. Then, according to an embodiment, as additional ultrasound data are acquired, additional frames or images generated from more-recently acquired ultrasound data are sequentially displayed. Additionally or alternatively, the ultrasound data may be stored temporarily in a buffer (not shown) during a scanning session and processed in less than real-time in a live or off-line operation. Some embodiments of the invention may include multiple processors (not shown) to handle the processing tasks. For example, a first processor may be utilized to demodulate and decimate the ultrasound signal while a second processor may be used to further process the data prior to displaying an image. It should be appreciated that other embodiments may use a different arrangement of processors.

Still referring to FIG. 1, the ultrasound imaging system 100 may continuously acquire ultrasound data at a frame rate of, for example, 20 Hz to 150 Hz. However, other embodiments may acquire ultrasound data at a different rate. A memory 120 is included for storing processed frames of acquired ultrasound data that are not scheduled to be displayed immediately. In an exemplary embodiment, the memory 120 is of sufficient capacity to store at least several seconds worth of frames of ultrasound data. The frames of ultrasound data are stored in a manner to facilitate retrieval thereof according to its order or time of acquisition. As described hereinabove, the ultrasound data may be retrieved during the generation and display of a live image. The memory 120 may comprise any known data storage medium.

Optionally, embodiments of the present invention may be implemented utilizing contrast agents. Contrast imaging generates enhanced images of anatomical structures and blood flow in a body when using ultrasound contrast agents including microbubbles. After acquiring ultrasound data while using a contrast agent, the image analysis includes separating harmonic and linear components, enhancing the harmonic component and generating an ultrasound image by utilizing the enhanced harmonic component. Separation of harmonic components from the received signals is performed using suitable filters. The use of contrast agents for ultrasound imaging is well known by those skilled in the art and will therefore not be described in further detail.

In various embodiments of the present invention, ultrasound information may be processed by other or different mode-related modules (e.g., B-mode, Color Doppler, power Doppler, M-mode, spectral Doppler anatomical M-mode, strain, strain rate, and the like) to form 2D or 3D data sets of image frames and the like. For example, one or more modules may generate B-mode, color Doppler, power Doppler, M-mode, anatomical M-mode, strain, strain rate, spectral Doppler image frames and combinations thereof, and the like. The image frames are stored and timing information indicating a time at which the image frame was acquired in memory may be recorded with each image frame. The modules may include, for example, a scan conversion module to perform scan conversion operations to convert the image frames from Polar to Cartesian coordinates. A video processor module may be provided that reads the image frames from a memory and displays the image frames in real time while a procedure is being carried out on a patient. A video processor module may store the image frames in an image memory, from which the images are read and displayed. The ultrasound imaging system 100 shown may comprise a console system, or a portable system, such as a hand-held or laptop-style system.

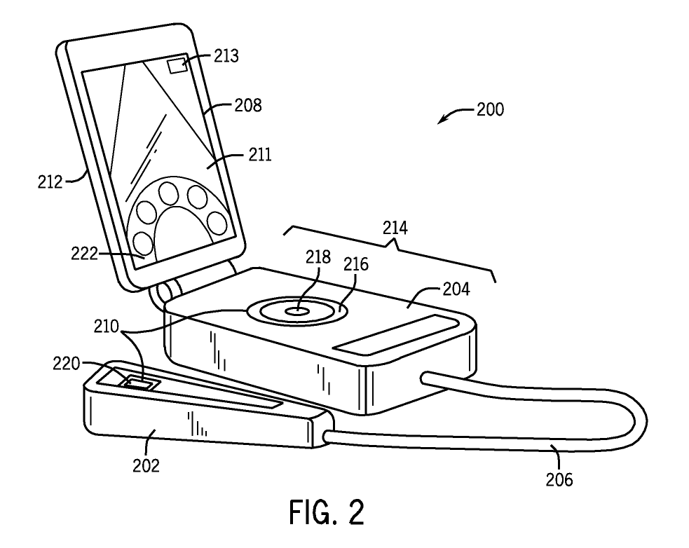

FIG. 2 is a schematic representation of hand-held ultrasound imaging system 200 in accordance with an embodiment. The hand-held ultrasound imaging system 200 includes a probe 202, a housing 204, and a cable 206 connecting the probe 202 to the housing 204. The hand-held ultrasound imaging system 200 includes a display screen 208 and a user interface 210. The display screen 208 of the exemplary hand-held ultrasound imaging system 200 may be used to show many types of ultrasound images including a live B-mode image 211. An indicator 213 is also displayed on the display screen 208 according to an exemplary embodiment. Additional information about the indicator 213 will be provided hereinafter. The display screen 208 is affixed to a folding portion 212 that is adapted to fold down on top of a main housing portion 214 during the transportation or storage of the hand-held ultrasound imaging system 200.

The user interface 210 of the hand-held ultrasound imaging system 200 comprises a rotary wheel 216, a central button 218, and a switch 220. The rotary wheel 216 may be used in combination with the central button 218 and the switch 220 to control imaging tasks performed by the hand-held ultrasound imaging system. For example, according to an embodiment, the rotary wheel 216 may be used to move through a menu 222 shown on the display 208. The central button 218 may be used to select a specific item within the menu 222. Additionally, the rotary wheel 216 may be used to quickly adjust parameters such as gain and/or depth while acquiring data with the probe 202. The switch 220 may be used to optionally show a target image as will be discussed in greater detail hereinafter. It should be appreciated by those skilled in the art that other embodiments may include a user interface including one or more different controls and/or the rotary wheel 216, the central button 218, and the switch 220 may be utilized to perform different tasks. Other embodiments may, for instance, include additional controls such as additional buttons, a touch screen, voice-activated functions, and additional controls located on the probe 202.

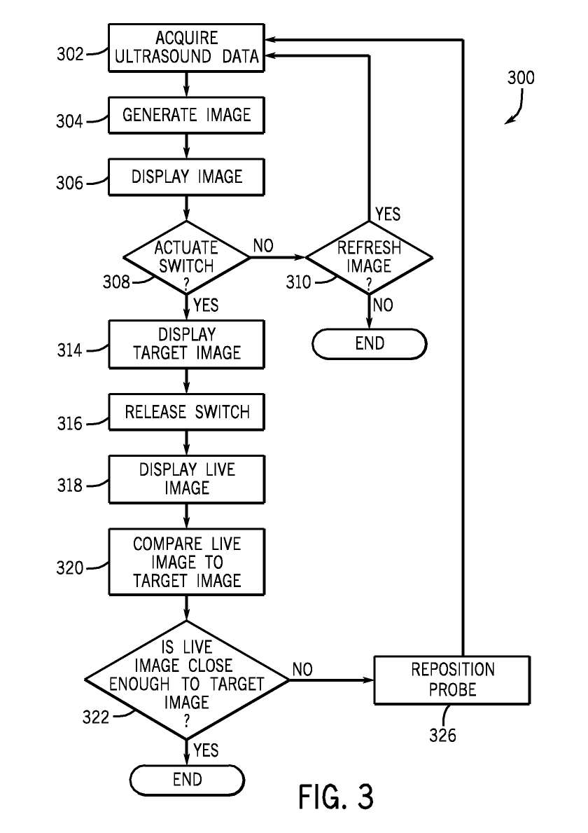

FIG. 3 is a flow chart illustrating a method 300 in accordance with an embodiment. The individual blocks represent steps that may be performed in accordance with the method 300. The technical effect of the method 300 is the display of a target image while in the course of acquiring ultrasound data.

According to an embodiment, the method 300 may be performed with the hand-held ultrasound imaging system 200 shown in FIG. 2. The method 300 may also be performed on other types ultrasound imaging system according to other embodiments. Referring now to both FIG. 2 and FIG. 3, at step 302 of the method 300, ultrasound data is acquired. Acquiring ultrasound data comprises transmitting ultrasonic sound waves from transducer elements in the probe 202 and then receiving reflected ultrasonic sound waves back at the transducer elements of the probe 202. For purposes of this disclosure, the term, “acquiring ultrasound data” may include acquiring enough data to generate one or more ultrasound images.

At step 304, an image or frame is generated from the ultrasound data acquired during step 302. According to an embodiment, the image may comprise a B-mode image, but other embodiments may generate additional types of images including Color Doppler, power Doppler, M-mode, spectral Doppler anatomical M-mode, strain, strain rate, and the like. The generation of an ultrasound image from ultrasound data is well known by those skilled in the art and, therefore, will not be described in detail.

At step 306, the image generated at step 304 is displayed on a display screen, such as the display screen 208 (shown in FIG. 2). At step 308, a user may actuate a switch. If the switch is not actuated at step 308, the method 300 advances to step 310. At step 310, a processor determines if the image should be refreshed. If a refreshed image is desired, the method 300 returns to step 302 where additional ultrasound data is acquired. Steps 302, 304, and 306 may be repeated many times while in the course of acquiring ultrasound data and displaying a live image. For example, during the display of a live image, steps 302, 304 and 306 may be repeated 100 or more times per minute. It should be appreciated by those skilled in the art that each time the method 300 cycles through steps 302, 304, and 306, the image displayed at step 306 is generated from ultrasound data acquired during a more recent time interval. According to other embodiments, the processes performed at steps 302, 304, and 306 may overlap. For example, while the processor 116 (shown in FIG. 1) is generating an image at step 304 based on previously acquired ultrasound data, the processor 116 may be controlling the acquisition of additional ultrasound data. Likewise, while the processor 116 is displaying the live image generated during step 304, the processor 116 may also be actively controlling the acquisition of additional ultrasound data. According to one embodiment, the acquisition of ultrasound data may occur more or less constantly while images are generated and displayed based on previously acquired ultrasound data. If a refreshed image is not desired at step 310, the method 300 ends.

Referring to step 308 in FIG. 3, if, according to an embodiment, the switch is actuated at step 308, the method advances to step 314 and a target image is displayed. The target image will be described in detail hereinafter. According to an embodiment, the switch may be the switch 220 (shown in FIG. 2). It should be appreciated that other embodiments may use a different type of user interface to control the display of the target image, including, but not limited to, buttons or switches located on an ultrasound console, buttons or switches located on the housing 204 (shown in FIG. 2), and a touch-screen. The actuation of the switch at step 308 sends an instruction to a processor, such as the processor 116 (shown in FIG. 1), to display a target image.

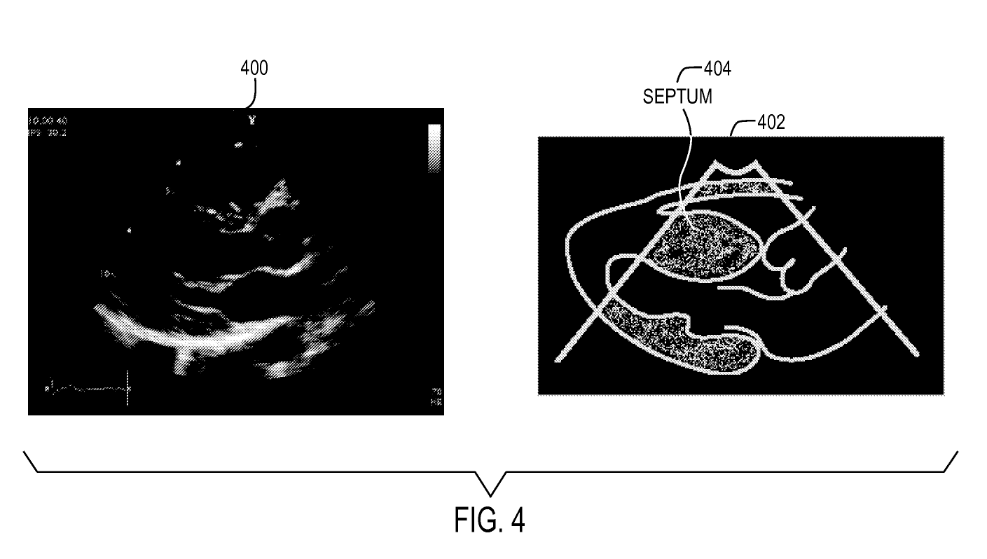

FIG. 4 shows schematic representation of both a live image 400 and a target image 402 in accordance with an embodiment. According to the embodiment shown in FIG. 4, the live image 400 shows a B-mode parasternal long-axis view of a patient's heart. The live image 400 is updated approximately 60 times per second according to an embodiment. Since it is updated so frequently, the live image 400 shows an almost real-time view of the ultrasound data being acquired by the ultrasound imaging system. It should be appreciated that the live image 400 may comprise anatomical structures other than a heart and that the view may be different according to additional embodiments.

The target image 402 comprises a standard view of the anatomical structure for which ultrasound images are desired. According to the embodiment shown in FIG. 4, the target image 402 comprises a parasternal long-axis view of a heart. It should be appreciated that the target image 402 is just one example of a standard view and that target images may comprise different anatomical structures and/or different standard views according to other embodiments. For example, the target images of other embodiments may comprise additional standard views of the heart, including a 4-chamber view, an apical long-axis view, and a 2-chamber view. Still other embodiments may include target images for anatomical structures other than the heart. The target image may include a gray scale image, such as a standard B-mode image, a Color Doppler image, or a Doppler image according to an embodiment. According to an embodiment where the target image comprises a Doppler image, the target image may be an exemplary Doppler waveform. Additionally, the target image may have the look and feel of a single frame of the live image according to some embodiments, or the target image may be a schematic representation of an image such as the target image 402. According to yet other embodiments, the target image may be either a static image or a dynamic image. As is well known by those skilled in the art, a static image does not change over time while a dynamic image includes multiple image frames, and, as such, may be used to demonstrate motion over a period of time. For example, a dynamic target image may be used to model the way the heart valves should move in a standard view. According to an embodiment, the target image 402 may also include an annotation 404. The annotation 404 labels the septum in target image 402. Annotations may be used to label other structures on a target image according to additional embodiments.

According to an embodiment, the processor 116 (shown in FIG. 1) may adjust one or more parameters of the target image 402 so that the live image 400 and the target image 402 are similar with respect to the one or more parameters. For example, it may be easier for a user to compare the live image 400 to the target image 402 if the parameter settings are generally similar between the live image 400 and target image 402. For example, the processor 116 may perform one or more image processing operations on the target image 402 to make it look more similar to the live image 400. These image-processing operations may include deforming the target image through various types of elastic deformations.

Referring to FIGS. 3 and 4, at step 316, the user releases the switch 220 (shown in FIG. 2). Then, at step 318, the live image 400 is displayed in response to the user releasing the switch 220. According to an embodiment, the display screen shows just the live image 400 when the user releases the switch 220. In other words, the target image 402 is only displayed when the user is actively pressing the switch 220. Other methods of switching between the live image 400 and the target image 402 may be used in other embodiments. For example, the user may press a button to switch from the live image 400 to the target image 402. The user may then press the same button a second time to switch back from the target image 402 to the live image 400. Different buttons or switches may be used to control the transition from the live image 400 to the target image 402 and the transition from the target image 402 to the live image 400 according to other embodiments. According to an embodiment, the target image 402 may be displayed in course of acquiring ultrasound data. For purposes of this disclosure, the term, “in the course of acquiring ultrasound data” includes the period of time during which ultrasound data is acquired to generate a plurality of images that are components of a live image. The term, “in the course of acquiring ultrasound data” may include both times where the ultrasound data is actively being acquired and times in-between the periods of active ultrasound data acquisition.

According to another embodiment, ultrasound data may be acquired during the time while the target image is displayed. Likewise, the processor 116 (shown in FIG. 1) may continue to generate refreshed images for the live image during the time while the target image is displayed. This way, the live image that is displayed represents an image generated from recently acquired ultrasound data, even during the time just after displaying the target image.

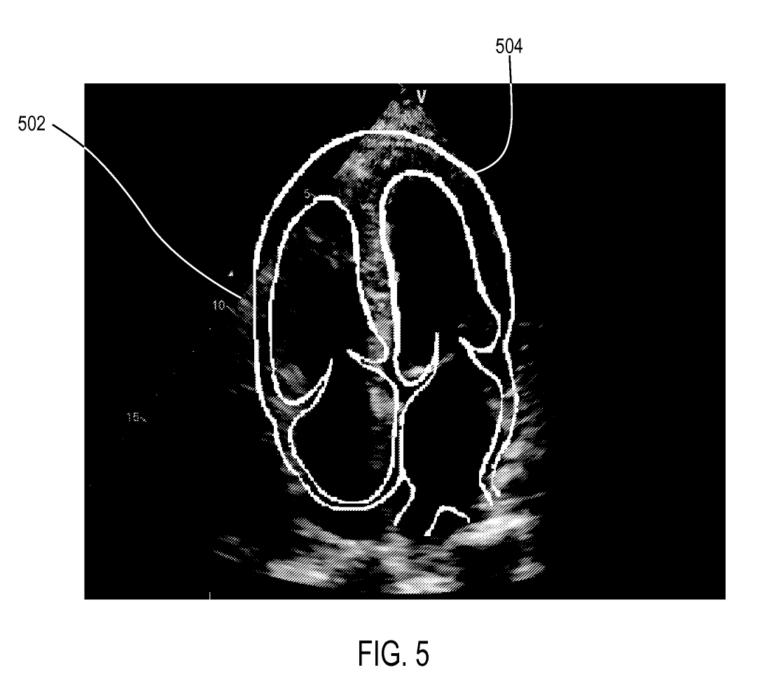

According to another embodiment, the method 300 may be modified so that both the live image and the target image are displayed at generally the same time. For example, FIG. 5 shows a schematic representation of a live image 502 with a target image 504 superimposed on top of the live image 502 in accordance with an embodiment. The live image 502 shows a B-mode parasternal short-axis view of a patient's heart. A target image 504 is superimposed on top of the live image 502. The target image 504 shows the relative orientation and positioning of the anatomy that would be typical for a parasternal short-axis view of the heart. The method 300 may be modified so that the target image is superimposed on top of the live image at step 314. Therefore, through the activation of a switch, the processor 116 (shown in FIG. 1) may selectively display either the target image 504 superimposed on the live image 502 or just the live image 502. It should be appreciated that the live image 502 is dynamic and being refreshed at a certain rate even while target image 504 is superimposed on the live image 502 according to an embodiment.

Referring back to FIG. 3 and FIG. 4, at step 320, the live image 400 is compared to the target image 402. It should be appreciated that the user may toggle back-and-forth between the live image 400 and the target image 402 multiple times in order to compare the live image 400 to the target image 402. The user may be trying to acquire data that results in an image that closely matches the standard view shown in the target image 402. Therefore, by adjusting one or more acquisition parameters and comparing the resulting live image 400 to the target image 402, the user may ultimately end up with a live image that closely matches the target image. One advantage of this embodiment is that it allows the user to iteratively adjust an acquisition parameter and compare the resulting live image 400 to the target image 402 multiple times in order to achieve a close match between the live image 400 and the target image 402. According to an exemplary embodiment, the user may use the target image 402 to adjust the acquisition parameter of probe position. As a result of comparing the live image 400 to the target image 402, the user is able to adjust the position of the probe in order to generate and display images that are consistent with a standard view of an anatomical structure according to a particular protocol that is represented in the target image 402.

According to other embodiments, the processor 116 (shown in FIG. 1) may automatically compare the live image 400 to the target image 402. The processor 116 may apply contouring to the live image 400 based on grey level thresholding in order to more easily make the comparison between the live image 400 and the target image 402. The processor 116 may, for example, make a determination of how closely the live image 400 matches the target image 402 based on a level of correlation between contours fitted to one or more frames of the live image 400 and the target image 402. The processor 116 may than display an indicator, such as the indicator 213 (shown in FIG. 2), on the display screen 208. The indicator 213 may comprise a status light. The status light may be green at times when the live image 400 closely matches the target image 402. The status light may be red at times when the live image 400 is significantly different from the target image 402. The status light may be yellow at times when the live image 400 correlates with the target image at a level in between the thresholds for the green light and the red light. Therefore, by observing status light, the user may be able to determine if the live image is approximately correct when attempting to acquire ultrasound data in order to generate an image showing a standard view.

According to an embodiment, the processor 116 (shown in FIG. 1) may calculate changes needed from the current probe position in order to position the probe in a new position that would result in the acquisition of additional ultrasound data that may be used to generate an image that more closely matches the target image. According to an embodiment, the instructions may include translating the probe in a given direction to a new location, changing the angle of inclination of the probe with respect to the patient's body, and rotating the probe in either a clockwise or counter-clockwise direction. The processor 116 may convey these instructions either as text on the display screen 208 (shown in FIG. 2) or as a series of verbal commands emitted through a speaker (not shown).

Referring to FIG. 3, according to other embodiments, the step 314 may be replaced with a step that involves the displaying of a dynamic target image. For the purposes of this disclosure, the term “dynamic target image” is defined to include a series of target images that are displayed in succession. Each of the target images that are part of the dynamic target image shows the anatomical structure at a different time. According to an embodiment, the dynamic target image may be used to show motion of an anatomical structure, such as the heart, from a standard view.

There are multiple ways that the user may use the dynamic image. According to one embodiment, the user may record or store a loop of images from the live image to create a dynamic image and then compare the dynamic image to a dynamic target image. The user may toggle between the stored loop of images and the dynamic target image multiple times to determine whether or not any corrections need to be made to the positioning of the probe in order to acquire a data that is closer to the standard view. The user may also directly compare the dynamic image to the live image. One advantage of this embodiment is that the user may make changes to the probe position in between checking the dynamic target image and see the effects of the change in almost real-time. According to yet another embodiment, the user may compare the live image to the dynamic target image on a frame-by-frame basis. That is, the user may compare a single frame from the live image to a single frame from the dynamic target image. According to an embodiment, the processor 116 (shown in FIG. 1) may use an image processing technique such as image matching in order to identify which image or images from the dynamic target image correspond to the current phase of the anatomical structure shown in the live image.

Referring back to FIG. 3, at step 322 the user determines if the live image is close enough to the target image. If the live image is close enough to the target image, then the method 300 ends. If the live image is not close enough to the target image, then the method 300 proceeds to step 326.

Referring to FIG. 3 and FIG. 4, at step 326 the probe is repositioned. The user may move the probe to a modified probe position based on the comparison of the live image 400 to the target image 402 performed during step 320. The user may position the probe so that the ultrasound data acquired at the modified probe position results in an image that is closer to the target image. After the probe has been repositioned, the method 300 returns to step 302 where additional ultrasound data is acquired at the modified probe position. The method 300 may involve iteratively repositioning the probe multiple times before the live image corresponds closely enough to the target image. The user may adjust other acquisition parameters according to additional embodiments.

It should be appreciated that while the method 300 was described as being performed with the hand-held ultrasound imaging system 200, the method 300 may also be performed with other types of ultrasound imaging systems including console ultrasound imaging systems and portable laptop-style ultrasound imaging systems.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

We claim:

1. A method of ultrasound imaging comprising:
acquiring ultrasound data of an anatomical structure;
generating a live image based on the ultrasound data;
displaying the live image;
displaying a target image of the anatomical structure; and
comparing the live image to the target image.

2. The method of claim 1, wherein said displaying the target image comprises displaying the target image in place of the live image.

3. The method of claim 1, wherein said displaying the target image comprises displaying the target image at generally the same time as the live image.

4. The method of claim 3, wherein said displaying the target image further comprises displaying the target image superimposed on the live image.

5. The method of claim 3, wherein said displaying the target image further comprises displaying the target image in a first portion of a display screen and displaying the live image in a second portion of the display screen.

6. The method of claim 1, further comprising calculating how well the live image matches the target image.

7. The method of claim 6, further comprising displaying an indicator representing how well the live image matches the target image.

8. The method of claim 1, wherein the target image comprises an annotation labeling a specific portion of the target image.

9. The method of claim 1, wherein said displaying the target image comprises displaying the target image while in the course of acquiring the ultrasound data.

10. The method of claim 1, further comprising acquiring additional ultrasound data based on said comparing the live image to the target image.

11. The method of claim 1, wherein the target image comprises a gray scale image, a Color Doppler image, or a Doppler image.

12. A method of ultrasound imaging comprising:
acquiring ultrasound data of an anatomical structure;
generating a live image based on the ultrasound data;
displaying the live image;
selectively displaying a target image of the anatomical structure while displaying the live image; and
comparing the live image to the target image in order to validate an acquisition parameter used to acquire the ultrasound data.

13. The method of claim 12, wherein the acquisition parameter is selected from a group consisting of width, depth, gain, and probe position.

14. The method of claim 12, further comprising modifying the acquisition parameter based on said comparing the live image to the target image.

15. The method of claim 12, wherein the target image is selectively displayed in response to the actuation of a switch.

16. The method of claim 12, wherein the target image comprises a dynamic image.

17. The method of claim 12, wherein the target image comprises a static image.

18. An ultrasound imaging system comprising:
a probe including a plurality of transducer elements;
a user interface;
a display screen; and
a processor operably connected to the probe, the user interface, and the display screen, the processor configured to control the probe to acquire ultrasound data of an anatomical structure, the processor configured to generate a live image from the ultrasound data, the processor configured to display the live image on the display screen, the processor configured to display a target image of the anatomical structure on the display screen in response to an input entered through the user interface.

19. The ultrasound imaging system of claim 18, wherein the user interface comprises a switch instructing the processor to display the target image on the display screen.

20. The ultrasound imaging system of claim 19, wherein the user interface comprises a switch positioned on the probe.

21. The ultrasound imaging system of claim 18, wherein the processor is further configured to superimpose the target image on top of the live image on the display screen.

22. The ultrasound imaging system of claim 18, wherein the processor is further configured to calculate how well the live image matches the target image.

23. The ultrasound imaging system of claim 22, wherein the processor is further configured to display an indicator on the display screen representing how well the live image matches the target image.

24. The ultrasound imaging system of claim 23, wherein the processor is further configured to convey an instruction regarding how to reposition a probe to acquire new ultrasound data that will result in a live image that more closely matches the target image.

25. The ultrasound imaging system of claim 27, wherein processor and the display screen are disposed in a housing that is adapted to be hand-held.
